(19) United States
(12) Patent Application Publication
(10) Pub. No.: US 2022/0206745 A1
(43) Pub. Date: Jun. 30, 2022

(54) RELATIONSHIP ANALYSIS UTILIZING BIOFEEDBACK INFORMATION

(71) Applicant: ONTOLEAD, INC., La Jolla, CA (US)

(72) Inventors: Firoozeh MOTAMEDI, La Jolla, CA (US); Shahrokh YADEGARI, La Jolla, CA (US)

(73) Assignee: ONTOLEAD, INC., La Jolla, CA (US)

(21) Appl. No.: 17/694,519

(22) Filed: Mar. 14, 2022

Related U.S. Application Data
(63) Continuation-in-part of application No. 16/135,989, filed on Sep. 19, 2018, now Pat. No. 11,275,557.
Application No. 16/135,989 is a continuation of application No. 15/644,601, filed on Jul. 7, 2017, now Pat. No. 10,089,074.
(60) Provisional application No. 62/360,186, filed on Jul. 8, 2016.

(51) Int. Cl. G06F 7/02; A61B 5/0533; A61B 5/16

(52) U.S. Cl. CPC G06F 7/02; A61B 5/167; A61B 5/0533

(57) ABSTRACT

Systems, methods, and computer software are disclosed for determining group dynamics. This can include receiving input data related to a particular group and determining, by a machine learning algorithm, a quantified group dynamic for the particular group. The machine learning algorithm can be trained with at least group information, user information, sensor data, and subjective evaluation data. A client device can generate an electronic indication of the quantified group dynamic.

### CROSS-REFERENCE TO RELATED APPLICATIONS

The current application claims priority to and is a continuation-in-part of pending U.S. patent application Ser. No. 16/135,989 entitled “RELATIONSHIP ANALYSIS UTILIZING BIOFEEDBACK INFORMATION,” which is a continuation of U.S. patent application Ser. No. 15/644,601 (now U.S. Pat. No. 10,089,074) entitled “RELATIONSHIP ANALYSIS UTILIZING BIOFEEDBACK INFORMATION,” which claims priority to U.S. Provisional Patent Application No. 62/360,186 filed Jul. 8, 2016 and entitled “RELATIONSHIP ANALYSIS UTILIZING BIOFEEDBACK INFORMATION,” the contents of each are hereby incorporated by reference in their entirety.

### BACKGROUND

Relationship decisions are made on many different levels and in many different situations. For example, when seeking out a romantic mate, people can decide based on gut feelings, attraction, or background checks. Relationship decisions also occur in the formation of groups, for example, for social or work-related purposes. The personalities of the individuals in the group, and how they relate to one another, will affect the operation of the group and the potential of the group to meet its goals.

### SUMMARY

Systems, methods, and computer software are disclosed for determining group dynamics. In one aspect, this can include receiving input data related to a particular group and determining, by a machine learning algorithm, a quantified group dynamic for the particular group. The machine learning algorithm can be trained with at least group information, user information, sensor data, and subjective evaluation data. A client device can generate an electronic indication of the quantified group dynamic.

In some variations, the quantified group dynamic can include objective results including one or more of task completion speed, group productivity, or group efficiency. The quantified group dynamic comprises subjective measures can include one or more of group health, group happiness, or group unity.

Other variations can include receiving, at the at least one programmable processor, updated input data, determining, by the machine learning algorithm, an updated quantified group dynamic, and generating, at the client device, an updated electronic indication of the updated quantified group dynamic. The receiving of the updated input data can include receiving ongoing sensor data.

In still other variations, the input data can include group information including one or more of a number of members or a length of time the group has been working together. The input data can include comprises user information including one or more of height, weight, or education level. The sensor data can include biofeedback data from a sensing device. The input data can include subjective evaluation data including an evaluated group dynamic determined based at least on the sensor data. The input data can include one or more of a room temperature, a room size, a time of day, or a noise level. The input data can also include task information including one or more of collaborative remote work or assembly line work.

In some variations, the sensor data can be from a galvanic skin sensor a nano-sensor inserted in the skin, EKG sensor, EEG sensor, temperature sensor, or fMRI. A sensing device that generates the sensor data can be contained in a watch or wristband.

Other variations can include determining an event window from the sensor data and adding an attribute to the sensor data in the event window, where the attribute is included with the input data.

In yet other variations, the particular group differs in one or more of a group size or group location from a training group from which the group information, the user information, the sensor data, and the subjective evaluation data was obtained.

In some variations, the operations can include performing an ongoing analysis of the input data to generate the quantified group dynamic, where the input data is received simultaneously with the ongoing analysis. Additional training of the machine learning algorithm can be performed during use of the machine learning algorithm when receiving the input data or determining the quantified group dynamic, the additional training based at least on the input data and subsequent subjective evaluation data. The machine learning algorithm can include one or more of a neural network or a deep belief algorithm.

Implementations of the current subject matter can include, but are not limited to, methods consistent with the descriptions provided herein as well as articles that comprise a tangibly embodied machine-readable medium operable to cause one or more machines (e.g., computers, etc.) to result in operations implementing one or more of the described features. Similarly, computer systems are also contemplated that may include one or more processors and one or more memories coupled to the one or more processors. A memory, which can include a computer-readable storage medium, may include, encode, store, or the like, one or more programs that cause one or more processors to perform one or more of the operations described herein. Computer implemented methods consistent with one or more implementations of the current subject matter can be implemented by one or more data processors residing in a single computing system or across multiple computing systems. Such multiple computing systems can be connected and can exchange data and/or commands or other instructions or the like via one or more connections, including but not limited to a connection over a network (e.g., the internet, a wireless wide area network, a local area network, a wide area network, a wired network, or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes in relation to particular implementations, it should be readily understood that such features are not intended to be limiting. The claims that follow this disclosure are intended to define the scope of the protected subject matter.

### BRIEF DESCRIPTION OF THE DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 is a diagram illustrating a simplified system for generating and presenting a compatibility score based on sensor data in accordance with certain aspects of the present disclosure;

FIG. 2 is a diagram illustrating an example of sensor data acquired from two users in accordance with certain aspects of the present disclosure;

FIG. 3 is a process flow diagram illustrating generating a compatibility score based on sensor data in accordance with certain aspects of the present disclosure;

FIG. 4 is a process flow diagram illustrating an example of certain manipulation and analysis of sensor data in accordance with other aspects of the present disclosure;

FIG. 5 is a diagram illustrating an example of phasic data and tonic data generated through discrete decomposition analysis in accordance with certain aspects of the present disclosure;

FIG. 6 is a diagram illustrating an example of the determination of an event value of exemplary phasic data in accordance with certain aspects of the present disclosure;

FIG. 7 is a diagram illustrating an example linear regression and an example determination of a variance of phasic data for two users in accordance with certain aspects of the present disclosure;

FIG. 8 is a diagram illustrating an example of a process for comparing the variance of sensor data for two users in accordance with certain aspects of the present disclosure;

FIG. 9 is a diagram illustrating an example of a process for comparing the linear regression analyses of sensor data for two users in accordance with certain aspects of the present disclosure;

FIG. 10 is a diagram illustrating a system for analyzing the sensor data for two users and providing a compatibility score in accordance with certain aspects of the present disclosure;

FIG. 11 illustrates a simplified system for generating and presenting a compatibility score based on sensor data and an evaluation of the sensor data received from an input device in accordance with certain aspects of the present disclosure;

FIG. 12 illustrates a simplified system for generating and presenting a quantified group dynamic determined by a machine learning system in accordance with certain aspects of the present disclosure; and

FIG. 13 illustrates an alternative embodiment for generating and presenting subjective measures determined by a machine learning system in accordance with certain aspects of the present disclosure.

### DETAILED DESCRIPTION

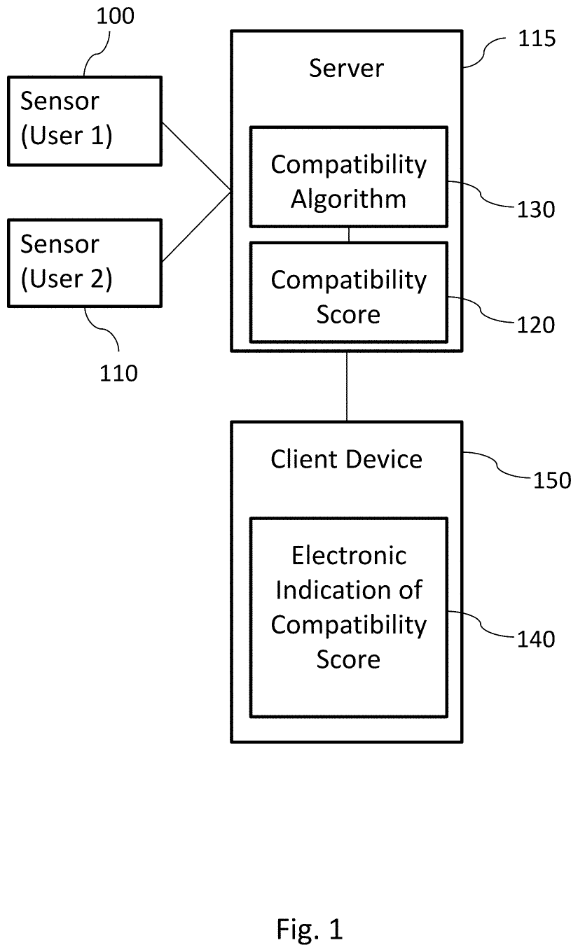

FIG. 1 illustrates a simplified system for generating and presenting a compatibility score 120 based on sensor data in accordance with certain aspects of the present disclosure. The present disclosure describes systems, methods, and computer software for gathering, storing, and analyzing sensor data from two or more individuals in, for example, the context of monitoring and maintaining relationships between them. As shown in FIG. 1, this can include executing a compatibility algorithm 130 at a server (or other computing device) with the sensor data being input into the compatibility algorithm 130.

The compatibility algorithm 130 can generate a compatibility score 120 based on the analyzed sensor data. The compatibility score 120 can be a metric indicative of the compatibility or harmony between the two or more individuals based on, for example, their respective sensor data and their compatibility preferences. The compatibility score 120 can be provided, for example, in the form of electronic data or as an electronic indication 140 (e.g., graphical, audio, textual, etc.) generated at a client device 150 (e.g., a smart phone, tablet computer, personal computer, etc.). Some non-limiting examples of uses and implementations of the system are provided below.

Match Evaluator—Users of the system can wear sensors whose sensor data can be continuously analyzed and uploaded to the cloud or other remote computing system through a wired or wireless network connection. Users can define their preferences for the type of people they are interested in. These preferences can include, for example, an indication of a lifestyle that can be extracted from sensor data (e.g., how active a person is, how excited do they get, what form of excitement ‘tonic/phasic’ they have). By matching the user's preferences to the sensor data of other users, the system can provide match suggestions to the user based on a compatibility score 120. Compatibility scores can be presented as, for example, a value between 0 and 1, which can be translated to, for example, “Great Fit”, “Good Fit”, “Not Bad”, “Little Fit”, or “No Fit”.

Personal Date Evaluator—Another example can include a user who has been wearing the sensors, and the sensor data has previously been uploaded to the system. The user can go on a date while wearing the sensor(s) or sensing device. The system can analyze and report the quality of the date based on the gathered sensor data, the user's lifestyle, and the user's preference for the type of reaction the user has selected as the best reactions to have on a date (this selection can define the level of excitement, the frequency of excitation, and the shape of features in the sensor data that correspond to various causes of excitement). The user can then input his or her evaluation of the date which can be used to train the system to learn about the user's preferences.

Date Match Evaluator for Two—In another implementation, two users who have been wearing sensors (and whose sensor data has been uploaded to the system), can go on a date while both are wearing the sensors and both have given each other access to their sensor data for the purpose of evaluation. The system can determine that the date has occurred based on GPS information obtained from the two sensors. The system can analyze and report the correlation of the recorded sensor data and compatibility of the two users with each other. If the users continue to have future dates, the system can analyze each of the dates, as well as record and monitor the evolution of the dates between the two users. The system can also report the quality of each date based on the ongoing evolution of the compatibility score 120 associated with each date.

Speed-Date Match Evaluator—A user wearing the sensor can go to a speed dating event (e.g., where many individuals come to meet each other and each couple will have specific amount of time to talk to each other, for example, around 5 to 10 minutes). The system can report about the quality of pairings individually to each user, or the system can cross analyze the compatibility score 120 generated from the sensor data between the participants who also have worn the sensors and are participants of the system described herein.

Relationship Harmony Measure—In another implementation, two users who have been wearing the sensing devices, and whose data have been uploaded regularly, can monitor their sensor data analysis and compare with their relationship partner. The sensors or other devices can also track their proximity to each other. The system can analyze and report on the correlation of their sensor data and their compatibility in their everyday life (e.g., when they are apart) in comparison to the times when they are close to each other. This can provide a measure of feedback on how their personal compatibility compares with their compatibility with people in general. The system can also provide feedback on the synchronization of their sensor data during their respective sleep cycles.

As used herein, sensor data, also sometimes referred to herein as “biofeedback data,” or “biofeedback” can include, but is not limited to, any type of measureable data relating to a physical, mental, or physiological state. The sensor data can include, for example, temperature, heart rate, electro-dermal activity (e.g., skin conductance), data relating to blood, urine, sweat, hormone, pheromone, saliva, catecholamine levels, metanephrine levels, measurements of minerals in sweat, and DNA. Other types of sensors can provide motion/acceleration measurements, muscle activity, brain activity (e.g., magnetoencephalography), other organ activity (e.g., EEG, EKG), body imaging (MRI, X-Ray), audio recording, visual images, video recording, positional information (e.g., GPS coordinates), and the like.

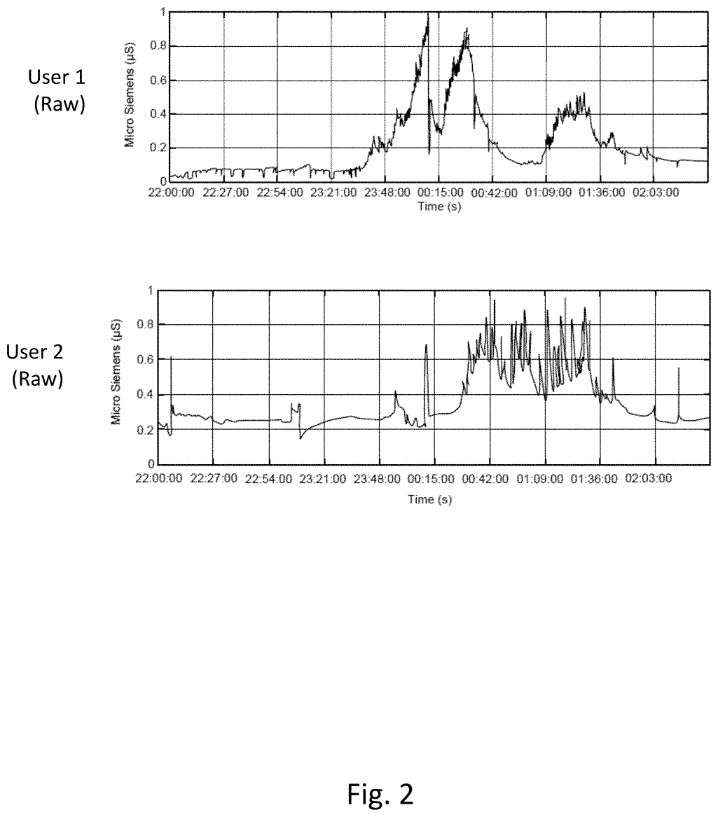

FIG. 2 illustrates an example of sensor data acquired from two users in accordance with certain aspects of the present disclosure. Sensor data can include, for example, analog or digital data received from the sensors or the sensing device. FIG. 2 shows an example time-resolved plot of sensor data acquired from sensors connected to a first user and a second user. In this example, the first sensor data and the second sensor data can be based on at least electro-dermal activity of the first user and the second user measured by the first sensor 100 and the second sensor 110. The gathering of the sensor data can be through one or more sensors operatively coupled to a sensing device or through other electronic devices. The sensing device can gather sensor data continuously, or on demand, from the subject.

In some implementations, a sensing device that contains one or more sensors can be, for example, a watch, wristband, electrode, or other external device that monitors or records sensor data for a user. The types of sensors that can be incorporated into the sensing device can include, for example, a photoplethysmography sensor, for measuring Blood Volume Pulse (BVP) and Heart Rate Variability (HRV), an accelerometer, an EDA sensor (or galvanic skin response GSR) sensor, and an infrared Thermopile sensor (for skin temperature). Other kinds of sensors can include electrochemical biosensors, optical biosensors, electronic biosensors, piezoelectric biosensors, gravimetric biosensors, or pyroelectric biosensors, and the like.

The sensor can be, for example, close to the body (either in direct contact or not), attached to the body, planted under the skin, inserted in the body (implants or nano-sensors), or inserted in the blood stream (nano-sensors). The sensing device can have its own internet connectivity capability and can send sensor data to a recipient device or computer as the sensor data is being acquired. The sensing device can also be connected to a network through a wire connection or wirelessly, for example with Bluetooth technology, to a local device. The local device can act as an intermediary or relay for transfer of sensor data to the server 115. Local devices can include, for example, a mobile phone or a tablet, with internet capabilities. The sensor data can be transferred to the cloud or other connected computing system in real-time when the sensor data is being gathered. The sensing device can also have its own computer memory and, when connection with the internet is established, it can send the gathered sensor data at a different time than the time that the sensor data is being gathered. In such implementations, the sensor data can be accumulated until a connection is established that allows the transfer of the sensor data to the desired computing system, for example, the server 115 illustrated in FIG. 1. The sensing device, the local mobile device, and or the server 115, can keep the sensor data in a raw format or analyze the sensor data as explained in greater detail below.

In some implementations, the sensing device or a complementary device can also record user-specified markers (e.g., identifying or time stamping events associated with the received sensor data).

Specific examples of complementary devices that can add information to the sensor data can include a clicker (e.g., a button or trigger) that can be on the sensor or another device (such as a cellphone). Single, double, and triple clicks can have different meanings, for example, a single click can mark an event selected by a user from a pre-selected list, a quick double click can be recorded as a positive event, and a quick triple click can be recorded as a negative event. A rotary button can allow the user to pick from a set of pre-selected evaluations such as: a very pleasant event—evaluation: 0.9, pleasant event—evaluation 0.7, average event—evaluation 0.5, negative event—evaluation 0.3, or a very negative event—evaluation 0.1. These evaluations can be implemented to provide weights to specific portions or features of the sensor data, as described further herein.

In other implementations, a clicker and rotary button can be used in conjunction with each other, a microphone can be used for the user to use voice to assign an evaluation to an event, or a video camera can be used for the user to use voice to assigning an evaluation to an event.

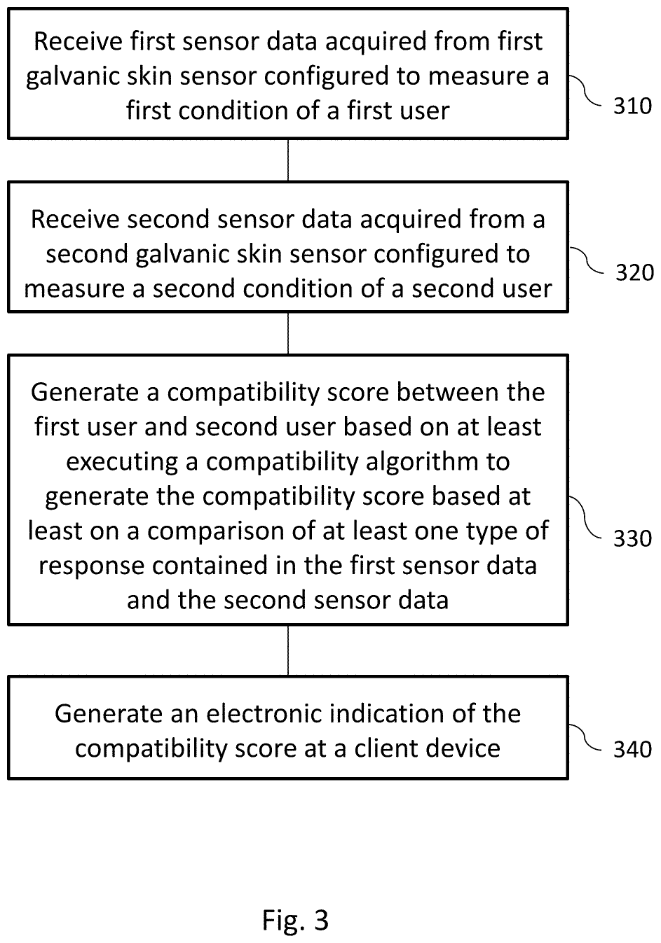

FIG. 3 is a process flow diagram illustrating generating a compatibility score 120 based on sensor data in accordance with certain aspects of the present disclosure. In one implementation, the systems, computer program products, and methods described herein can include, at 310 receiving first sensor data acquired from a first sensor 100 monitoring a first user. In some implementations, the monitoring of the user can be performed in real-time and generally simultaneous with the ongoing analysis of the sensor data. In other implementations, the monitoring can have been performed previously with the sensor data stored and/or transmitted to a server or other computing device that can analyze the data as described herein.

At 320, second sensor data can be received that was acquired from a second sensor 110 monitoring a second user.

At 330, a compatibility score 120 between the first user and the second user can be generated. The generating can include executing a compatibility algorithm 130 to generate the compatibility score 120 based at least on a comparison of at least one type of response contained in the first sensor data and the second sensor data.

At 340, a client device 150 can generate an electronic indication 140 of the compatibility score 120.

A sensor, as used herein, refers to the particular sensor that enables the sensing device to function in its prescribed manner. For example, when the sensing device is an electrodermal device or galvanic skin response sensor configured to measure skin conductance (which can change in response to an increase or decrease in a person's sweating), the sensor can be two electrical contacts having a potential difference. The amount of current transferred between the two electrical contacts comprising sensor can be related to the change in the skin conductance. As another example, a thermometer can be a sensing device that includes a thermocouple as a sensor.

A sensing device, as used herein, refers to a device that is directly connected to or operatively coupled with one or more sensors. The sensing device can include, for example, any combination of receivers, transmitters, and transceivers that can be configured to transmit or receive data to or from the sensing device. Examples of sensing devices can include, for example, a smartwatch with sensing capabilities, a medical device configured to acquire physical data about a person, a camera that can view a person or a portion of a person's anatomy, a microphone that can be used to generate audio recordings of a person, or the like.

As used herein, a “response” or “type of response” contained in the sensor data refers to a discrete and quantifiable aspect of the sensor data that can be identified in the raw sensor data or extracted or derived from the raw sensor data or processed sensor data through data analysis. Examples or responses or types of responses that can be present in sensor data can include, but are not limited to, a tonic response, a phasic response, a high-frequency response, a low-frequency response, a change in the amplitude or shape of the sensor data, or the like. Certain types of responses can be extracted from processing the sensor data, for example by applying low-pass filtering to extract a low-frequency response.

Terms such as “objective analysis,” “objective data,” or the like, as used herein, generally refer to analysis and data based purely on actual data acquired from a sensor or the analysis of same. In contrast, terms such as “subjective analysis,” “subjective data,” or the like, as used herein, generally refer to analysis and data that has some component that has been defined by the user. In this section, features relating to “objective analysis” are discussed. Later, features relating to “subjective analysis” are discussed.

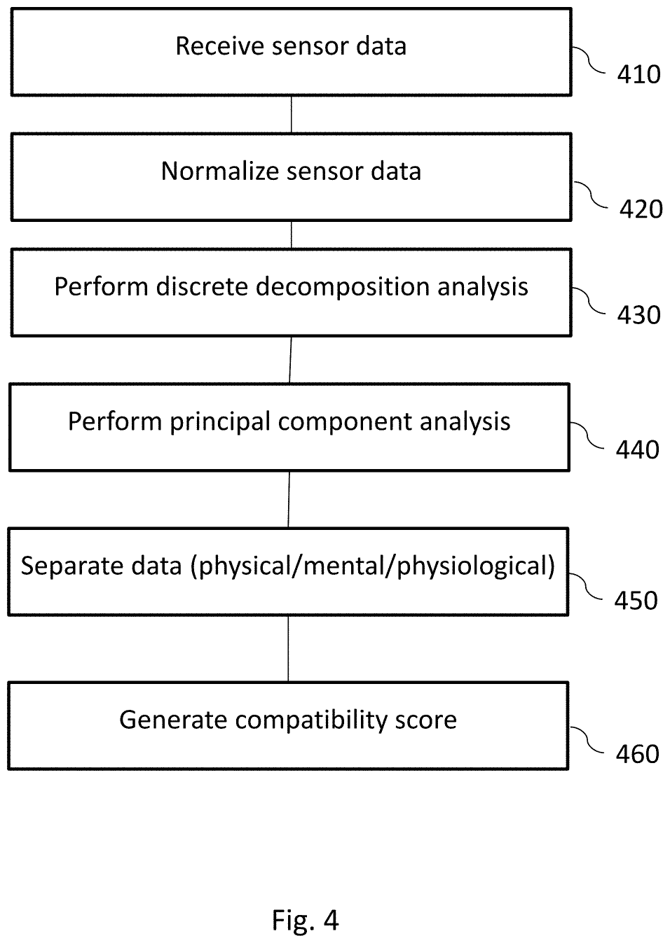

FIG. 4 is a process flow diagram illustrating an example of certain manipulation and analysis of sensor data in accordance with other aspects of the present disclosure. Analysis of the sensor data can be performed by the compatibility algorithm 130 to generate a compatibility score 120 relating to the users providing the sensor data. The analysis described below can include, for example, at 410, receiving the sensor data, at 420, normalizing the sensor data, at 430, performing discrete decomposition analysis to identify the types of responses present in the sensor data, at 440, performing principal component analysis to exclude some sensor data that is not as relevant as other types of sensor data, at 450, separating the sensor data into categories such as physical data, mental data, or physiological data, and, at 460, generating the compatibility score.

In some implementations, the sensor data can be normalized to a specified range (e.g., 0-1, 1-10, 1-100, −1 to +1), etc.). Such a normalization can be used to account for differences in diagnostic sensitivity, the individual response of a person to an event, the particular differences in a person's response in terms of a particular type of sensor data, or the like. For example, the sensor data from a first user and a second user can be normalized to both be in a range of 0-1 in order to facilitate later analysis. Such normalization can be applied at any stage of the analysis.

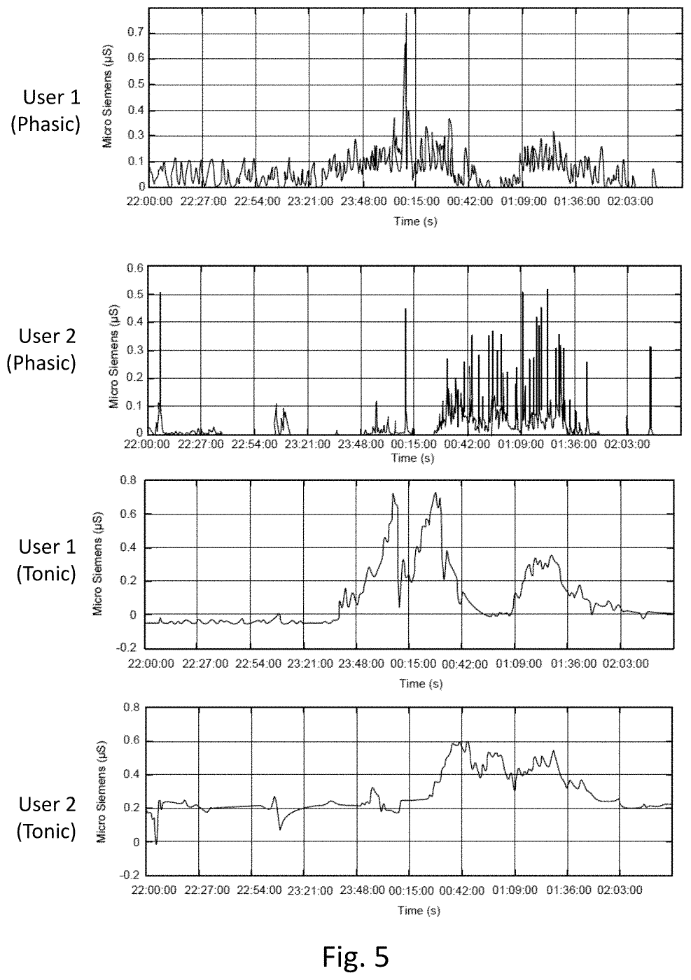

FIG. 5 is a diagram illustrating an example of phasic data and tonic data generated through discrete decomposition analysis in accordance with certain aspects of the present disclosure. The sensor data can also be separated into or otherwise identified as representing at least one type of response (e.g., a fast response and a slow response). A fast response (which can include a phasic response), can be sensor data that reflects a fast physiological response to an event or stimulus. In the specific case of a phasic response, the response captured in the sensor data is generally not present or quickly diminishes once the stimulus is removed. For slow (or tonic) responses, the sensor data can include a response with a slow onset and/or decay time after the event and or after the stimulus has been removed. In some implementations, the sensor data can first be analyzed to extract the tonic data by smoothing the sensor data to provide a baseline. The baseline can then be subtracted from the sensor data to provide the phasic data. The degree of smoothing applied to the sensor data can be predefined in the system or can be set by a user. In other implementations, a negative deconvolution can be applied to the sensor data to separate the tonic data and the phasic data.

Accordingly, in some implementations, the at least one type of response can include a tonic response and a phasic response. The first sensor data can include first phasic data and first tonic data, and the second sensor data can include second phasic data and second tonic data. The phasic data can correspond to the phasic response and the tonic data can correspond to the tonic response. The operations performed in analyzing the sensor data can include separating, by at least a first discrete decomposition analysis, the first sensor data into a first tonic response and a first phasic response. Similarly, the operations can also include separating, by at least a second discrete decomposition analysis, the second sensor data into a second tonic response and a second phasic response.

Based on the presence of a tonic response and a phasic response, first tonic data and second tonic data can be extracted from the first sensor data and the second sensor data. Similarly, first phasic data and second phasic data can be extracted from the first sensor data and the second sensor data. The comparison of sensor data between the users can be further based on the first tonic data the second tonic data, the first phasic data and the second phasic data. As described herein, the comparison can be used to generate a compatibility score 120.

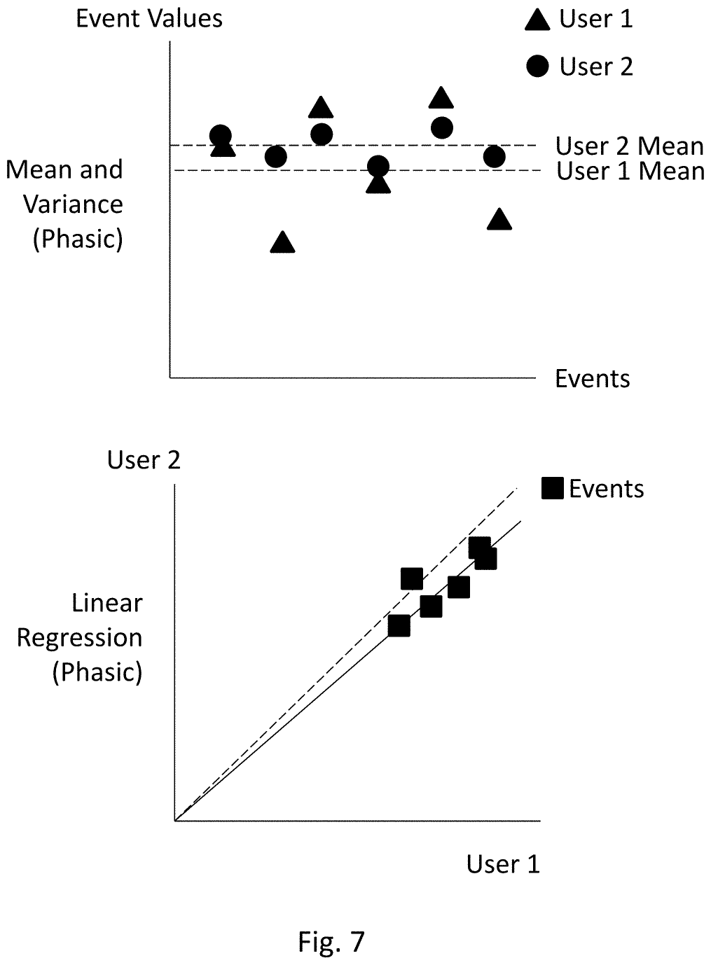

More particularly, some implementations can include performing statistical analysis on one or more of the tonic data, the phasic data, or any combination thereof to generate the compatibility score. Examples of statistical analysis such as linear regression and calculation of variance are described with reference to FIG. 7.

In some implementations, the tonic data can be considered. For example, the compatibility score can be generated by at least performing statistical analysis of first tonic data and second tonic data extracted from the first sensor data and the second sensor data.

In other implementations, the phasic data can be considered. For example, the compatibility score can be generated by at least performing statistical analysis of first phasic data and second phasic data extracted from the first sensor data and the second sensor data.

In yet other implementations, a combination of tonic and phasic data can be considered. For example, the compatibility algorithm 130 can perform a first statistical analysis of first tonic data and second tonic data extracted from the first sensor data and the second sensor data. Additionally, the compatibility algorithm 130 can also perform a second statistical analysis of first phasic data and second phasic data extracted from the first sensor data and the second sensor data. The compatibility score 120 can be based at least on the first statistical analysis and the second statistical analysis. Some specific implementations of these statistical analysis methods are described in further detail below.

To improve the correlation of the sensor data (which can include many types of measurements as discussed above) with a particular event, some types of sensor data can be removed or excluded from later analysis. Some implementations of the current subject matter can include filtering, by at least applying principle component analysis, the sensor data to exclude one or more types of the sensor data. The filtering can remove at least one component of the sensor data as identified by the principle component analysis. For example, if the sensor data included heart rate and GPS position, a given event such as a pain response may be more correlated with the user's heart rate than the GPS position provided by another sensor. The filtering can then remove the component (e.g., type of sensor data) that is not primarily correlated with the particular event that generated the sensor data.

The exclusion of sensor data can be performed by, for example, one or more of Principal Component Analysis (PCA), Principal Component Regression (PCR), Partial Least Squares Regression (PLSR), Sammon Mapping, Multidimensional Scaling (MDS), Projection Pursuit, Linear Discriminant Analysis (LDA), Mixture Discriminant Analysis (MDA), Quadratic Discriminant Analysis (QDA), and Flexible Discriminant Analysis (FDA).

In some implementations, the system can separate the sensor data into physical, physiological, and mental data. As used herein, “physical” data can be defined as all the changes in the sensor data that is generated through physical activity of the body. For example, some types of the sensor data of a person will increase while exercising (e.g., heart rate, sweating, and respiration). As used herein, “physiological” data is defined as all the data and fluctuations that are related to the bodily functions, but separate from intentional activity by a person. Examples of physiological data can include, how circadian clock affects our daily cycle, or how a women's monthly period can affect their sensor data, or how taking certain regular medication can affect the overall sensor data. “Mental” data is defined herein as the sensor data that can be generated through emotional and intellectual engagement of the user (often by the sympathetic nervous system) with events. Such data separation can be used to further filter or restrict the types of sensor data analyzed to determine the compatibility score 120.

In some implementations, after the discrete decomposition analysis is completed the sensor data can be analyzed with principal component analysis. All data that is highly correlated with sensors that measure physical activity (such as an accelerometer) can be classified as “physical data.” Data can be aggregated over time to learn about physiological aspects of a user's body, and all the data that match this category will be classified as “physiological data.” All the other data that is neither physical nor physiological can be classified as “mental data.” These classifications of sensor data can be stored and, in some implementations, used at least in part as the basis for calculating the compatibility score 120.

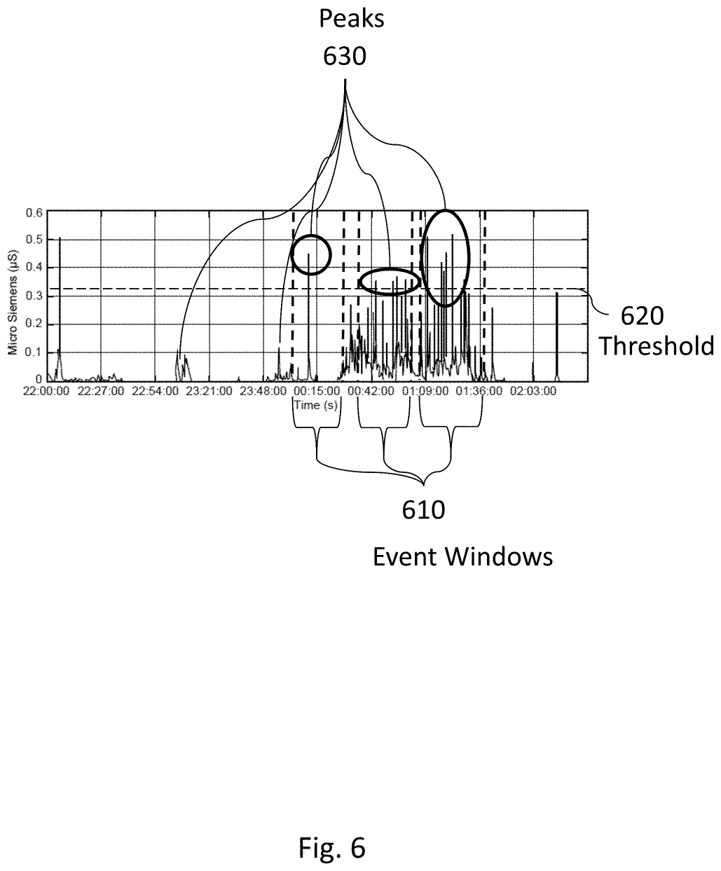

FIG. 6 is a diagram illustrating an example of the determination of an event value of exemplary phasic data in accordance with certain aspects of the present disclosure.

As used herein, an “event window” is any time window in the sensor data specified either by a user (such as providing a subjective evaluation of an event or interaction) or algorithmically (such as automatically by the systems herein or a separate system configured to identify event windows). The event window 610 can generally correspond to an event, for example a date, a greeting, a conversation, or the like. The sensor data can sometimes include a number of features during the event window 610 that can be further analyzed to quantify a specific type of reaction of a user to the event.

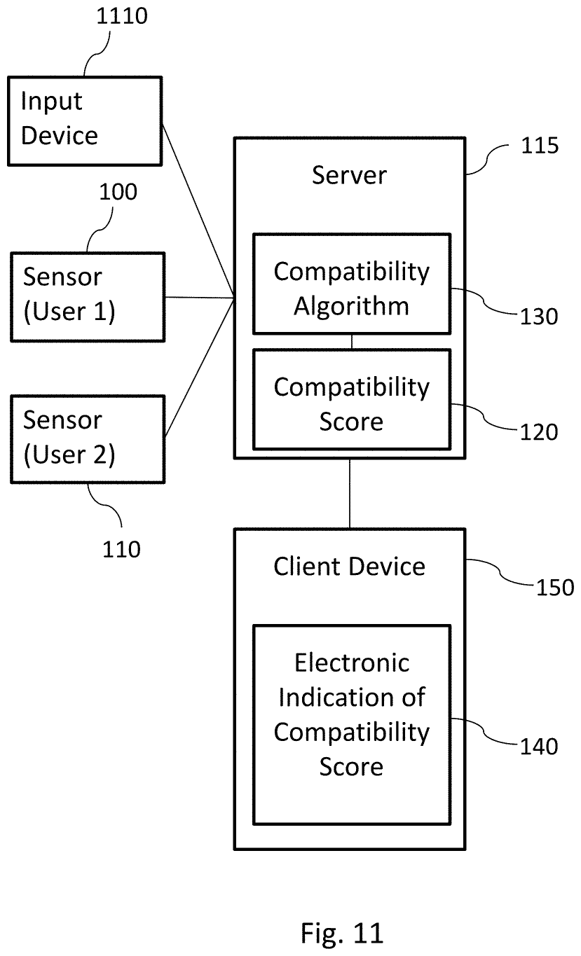

In some implementations, an attribute can be added to the sensor data in the event window 610. The attribute can include a name or other identifier of the event, time-stamps (e.g., noting that a particular feature of the sensor data occurred at a specific time or time window), or other similar metadata associated with the sensor data in the specified event window 610. Some attributes can be defined by the user through an input device and some attributes and event windows can be detected algorithmically by clustering features in the sensor data. Use of an input device is described further with reference to FIG. 11. The user can also define the event window 610 at the input device, or the system can automatically determine an appropriate event window 610.

As used herein, an “input device” 610 refers to any sort of mechanical or computing device that can receive input from a user and convert the input to a signal or data that can be interpreted by the compatibility algorithm. For example, an input device can be a smartphone, laptop computer, personal computer, complementary device, button, clicker, or the like.

Events that are represented by the sensor data can be rated, characterized, or generally quantified by analyzing features of the sensor data. These features can include the number of peaks above a particular threshold, the amplitude of the sensor data, particular features of the sensor data (e.g., shape of peaks, rise times, etc.), or the like. In some implementations, the event value (or intensity, amplitude, etc.) corresponding to an event window 610 (corresponding to a particular event) can be determined, by at least clustering a number of peaks in the sensor data to generate an event value for a portion of the sensor data. As used herein, “clustering” refers to identifying and grouping one or more features of the sensor data for further analysis, such as calculating an event value that corresponds to the clustered features of the sensor data. The event value can generally reflect the intensity of the event as it relates to a particular type of data. For example, an event can cause a strong response in the phasic data but not as strong in the tonic data. To determine the event value, the clustering can include filtering the sensor data to identify a number of peaks in the sensor data during the event window 610 that have an amplitude of at least a threshold value 620. The event value can be generated based on the number of peaks in the event window 610. These event values can serve as the basis for any of the statistical analysis applied to the sensor data. For example, as described further herein, the event value can be used to generate ratios of tonic or phasic data, or can be compared between two users with linear regression analysis.

Returning to the example of FIG. 6, the compatibility algorithm 130 (or the user) can define an event window 610 as spanning a particular time window, here shown by the dashed vertical lines in the plot and the brackets. During any of these event windows 610, a threshold value 620 for the phasic data can be applied. The number of peaks 630 that exceed the threshold value 620 can be counted and used to determine the event value. In this example, the first event window 610 contains a single peak 630 that exceeds the threshold 620. The latter two event windows 610 each contain several peaks 630 that exceed the threshold 620. In general, the more peaks 630 above threshold and the higher the amplitude of the peaks 630, the stronger the event is considered to be as it relates to the type of sensor data. In other implementations, the event value can be calculated relative to a baseline, and can be positive or negative, with the threshold value being below the baseline for “negative” peaks. In some implementations, the system can assign to the event value, or the sensor data in the event window 610, a timestamp that coincides with the midpoint of an event window 610.

FIG. 7 is a diagram illustrating an example linear regression and an example determination of a variance of phasic data for two users in accordance with certain aspects of the present disclosure. In some implementations, the processed sensor data (e.g., tonic data, phasic data, and event value) can be statistically analyzed by the compatibility algorithm 130 to provide a quantitative measure of compatibility. One type of statistical analysis that can be applied can include comparing the variance in a particular data type, or set of event values, between two users. In this example, user 1 shows a larger variance in phasic data than user 2. Also, in the example of FIG. 7, user 1 shows a less phasic response as compared to user 2, based on the mean of event values identified in their phasic data.

Also referring to the example of FIG. 7, a comparison can be made between the event values of the phasic events common to both users (shown by squares in the lower plot). In this example, a scatter plot of the phasic events shows that while there is more variation in the phasic data in user 2 than in the phasic data of user 1, the best fit (solid) line through both sets of phasic data, determined by linear regression, are closely matched in slope. If the best-fit line was along the dashed line, this would indicate a perfect linear correlation between user 1 and user 2, in terms of the plotted events. This can suggest that user 1 and user 2, on average, exhibit a good degree of harmony and compatibility. Either or both of the linear regression and variance determination can be used to calculate the compatibility score 120.

The particular examples described herein for the specific analysis of tonic and/or phasic data are not intended to be excluding of other implementations that involve other types of sensor data. For example, variance calculations and linear regression analysis can also be applied to heart rate sensor data, breathing rate sensor data, brain wave sensor data, etc.

In some implementations, compatibility algorithm 130 can generate the compatibility score 120 based on two features of the analyzed sensor data. The first feature can be a comparison of the ratios of the variance in a particular type of data between two users. The second feature can be a comparison of the linear regressions of a particular type of data between two users.

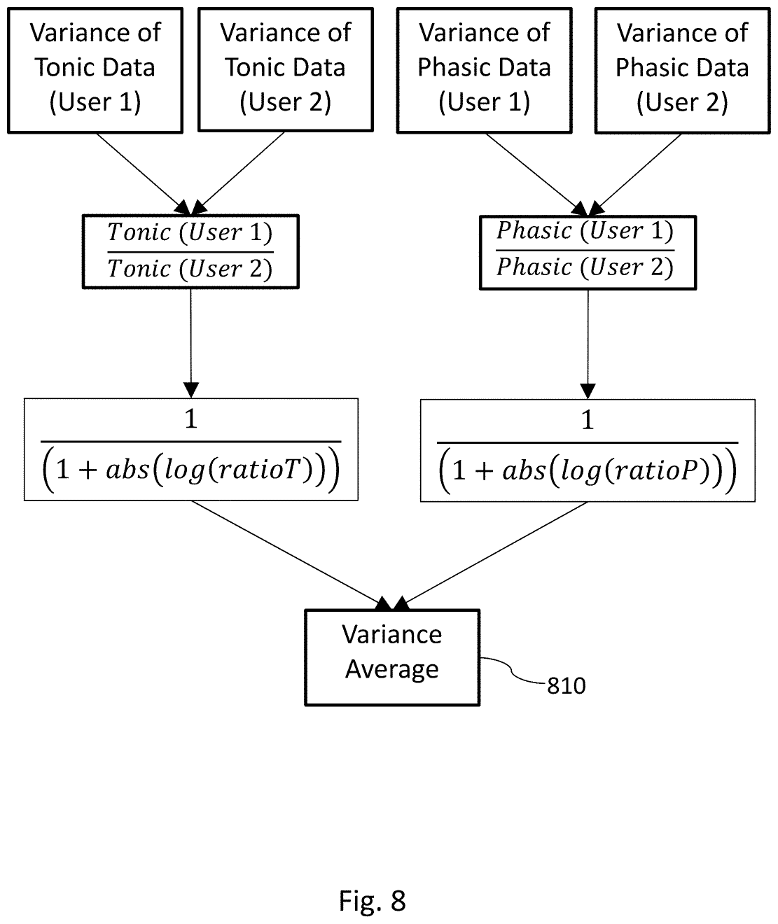

FIG. 8 is a diagram illustrating an example of a process for comparing the variance of sensor data for two users in accordance with certain aspects of the present disclosure. The variance of a desired subset of data (e.g., tonic data or phasic data) for two users can be expressed as a ratio. This ratio can generally reflect the degree of harmony or compatibility between two users, when considering that particular type of data. For example, two users who are compatible in their tonic data may have a ratio close to one. Two users who are less compatible may have a ratio significantly larger than one or smaller than one. In some implementations, the compatibility algorithm 130 can calculate a first ratio (ratioT in FIG. 8) of a tonic variance between the first user and the second user and a second ratio (ratioP in FIG. 8) of a phasic variance between the first user and the second user. In some implementations, the ratios can then be input to the formula,

$$\frac{1}{( 1 + abs ( log ( ratio ) ) )} . \qquad ( 1 )$$

Eqn. 1 provides a number between 0 and 1. Because there can be more than one metric (e.g., the result of Eqn. 1) for comparison, an average of the available metrics can be calculated. In the example of FIG. 8, the two numbers resulting from Eq. 1 for the tonic and phasic data can be averaged and the resulting “variance average” 810 can reflect a degree of similarity between the variance in the sensor data between the two users. The variance average 810 can be used in the final determination of the compatibility score 120.

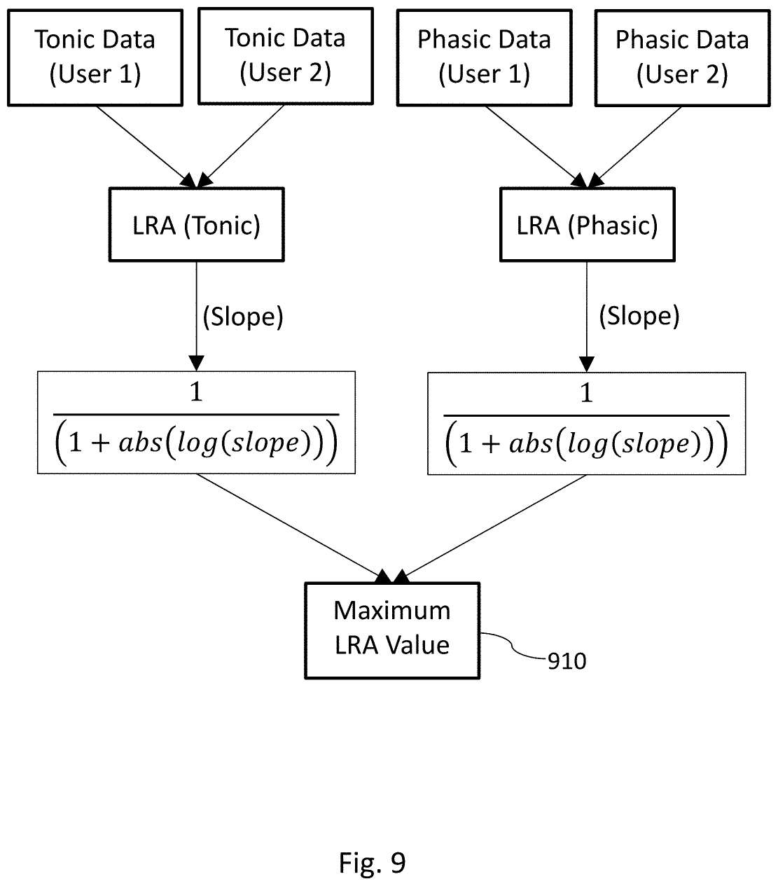

FIG. 9 is a diagram illustrating an example of a process for comparing the linear regression analyses of sensor data for two users in accordance with certain aspects of the present disclosure. In some implementations, generating the compatibility score 120 can include performing a linear regression analysis of the tonic data and the phasic data of the first user and the second user. The linear regression analysis can compare the event values of tonic data of a first user with the event values of the tonic data of a second user to generate a best-fit lines through their respective data sets. Similar to the evaluation of the ratios of the variance, the slopes resulting from the linear regression analysis for the first user and the second user can be evaluated with Eqn. 1. This can generate two numbers between zero and one that can represent the degree of correlation or harmony based on the linear regression analysis of the same types of sensor data (e.g., tonic or phasic). The greater of these two numbers can be stored by the system and is referred to herein as the “maximum LRA value” 910.

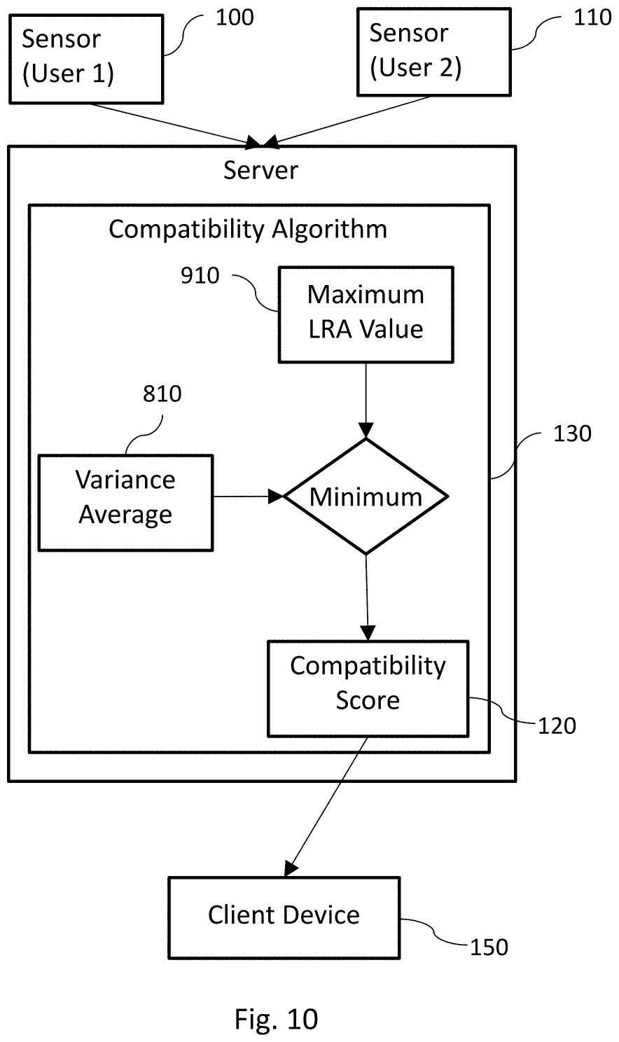

FIG. 10 is a diagram illustrating a system for analyzing the sensor data for two users and providing a compatibility score in accordance with certain aspects of the present disclosure.

Once the variance average 810 and the maximum LRA value 910 of the corresponding types of sensor data (e.g., tonic or phasic) are calculated, the minimum of the two can be provided as the compatibility score 120. In this way, the compatibility score 120 can be proportional to an average of the first ratio and the second ratio, and the compatibility score 120 can also be proportional to a measure of agreement in the linear regression analysis for the first user and the second user. The linear regression analysis between the event values of a first user and a second user or group are most in agreement when the line through their event values is along the 45 degree line of their linear regression plot (e.g., the dashed line in FIG. 7). This proximity to the 45 degree line can be one type of measure of agreement of the event values reflected in the linear regression analysis. The further that the linear regression is from this ideal, the lower the LRA value.

Because the system includes a decision point (e.g., determining a minimum) the compatibility score is only proportional to the variance average 810 when the variance average is less than maximum LRA value 910. Conversely, when the maximum LRA value 910 is less than the variance average 810, the compatibility score 120 is proportional to the maximum LRA value 910. By returning the minimum of the compatibility based on the linear regression analysis of the sensor data and the variance of the sensor data, the compatibility algorithm 130 can provide a lower bound quantification of the compatibility or harmony between the two users. In other implementations, the maximum can be used to provide an upper bound quantification of the compatibility between two users.

Other implementations can perform similar operations as the above but, for example, with other expressions instead of Eqn. 1. The above example is not intended to be limiting. In general, the compatibility algorithm 130 can take a collection of sensor data and separate the sensor data into components that can be present in the sensor data (e.g., tonic and phasic) and then analyze the components (or the raw sensor data itself) to determine similarities and differences in the raw or processed sensor data for the two users. The degree of similarity or difference can then be returned by the server 115 as a compatibility score 120 for presentation, storage, or display on a client device 150.

FIG. 11 illustrates a simplified system for generating and presenting a compatibility score 120 based on sensor data and an evaluation of the sensor data received from an input device 1110 in accordance with certain aspects of the present disclosure.

In some implementations, users can input, through an input device 1110, information relating to the analysis for themselves or another user. The system can receive an evaluation from the input device 1110 where the user characterizes the first sensor data or the second sensor data in an event window 610. The compatibility score 120 can then be based in part on the evaluation. For example, a user can review the sensor data and at various points in the sensor data (e.g., peaks, valleys, or other features related to an event) can add, through the input device 1110, an evaluation of the event. The evaluation can include, for example, a numerical rating, a verbal description (e.g., “good,” “average,” or “bad”), a video of the user reacting to an event, or the like. In the case of verbal or video capture of a user's evaluation, voice and/or image recognition software can be used to convert the input from the input device 1110 to an evaluation that can be interpreted by the compatibility algorithm 130. The quantified and/or converted evaluation can be used to, for example, modify event values, adjust tonic or phasic ratios, train a machine learning algorithm (when the compatibility algorithm 130 is based in part on a machine learning algorithm), or the like.

User preferences can be selected and entered by the user at the input device 1110. These user preferences can include specifying a kernel used in the discrete decomposition analysis for defining the shape of the impulses that are reflected in the sensor data. In other implementations, the time window used for clustering can be specified by a user. Also, in other implementations, the user can specify multipliers or weights for specifying the contribution of the mean of the analyzed sensor data or the variance of the analyzed sensor data.

A user can, through an input device 1110 such as a clicker (a device that can be toggled to indicate that an event occurred), a microphone, or a camera, specify a type of event that corresponds to the sensor data (e.g., a date, a greeting, a farewell, etc.). The type of event can be added to the sensor data as an attribute or other form of metadata that associates the type of event with some portion of the sensor data that corresponds to the event. For example, a spike in the sensor data for a heart rate sensor can be associated with a type of event by adding a text attribute such as “pain” to the portion of the sensor data (or the event window 610 as discussed in FIG. 6) that indicates the increased heart rate. Other types of attributes that can be added to the sensor data can include a time, location, intensity, or the like. These attributes can be used to further classify and provide a basis for correlation of the sensor data. For example, the compatibility score 120 can be generated based at least on a comparison of two persons' sensor data that corresponds to a physical activity such as jogging, where the sensor data for the two users have attributes that identify the portions of the sensor data acquired when they were jogging.

In some implementations, users can define which aspects of an event or features of the sensor data will bear more weight in the analysis process. The compatibility algorithm 130 can use data analysis to determine the existence of a desired feature in a potential match. This can include, for example, the system receiving user input from the first user specifying a desired feature of the second user. As a result, the compatibility algorithm 130 can increase the compatibility score 120 when the second sensor data reflects the desired feature.

These desired features can reflect a desired physiological, emotional, or personality trait in a potential match. For example, a first user may want a certain degree of calmness or excitability in a match. In some cases, a calmness can be reflected in or related to the amount of tonic sensor data acquired from a second user as compared to the amount of phasic sensor data. In such implementations, a user can configure the compatibility algorithm 130 to weight the tonic data more heavily than the phasic data when determining compatibility. In another implementation, a user can configure the compatibility algorithm 130 to weight the linear regression analysis more than the variance analysis. These weightings can be entered through a user interface, supplied by a configuration file read by the compatibility algorithm 130, or the like.

Therefore, in some implementations, a first user can select, at a graphical interface of an input device, the desired feature of the second user from a predefined list. The predefined list can include, for example, a calmness and an excitability. In this example, the compatibility score can be based at least on an objective determination that the second user has the desired feature through analysis of the second sensor data. The calmness or the excitability can be determined from the objective determination by the compatibility algorithm 130 based at least on a minimum mean or a maximum variance of the tonic data or the phasic data of the second user.

The above example of a compatibility algorithm 130 providing a compatibility score 120 was provided in terms of a single type of sensor data (e.g., only electro-dermal activity). Other implementations can, in general, be generalized to include an arbitrary number of types of sensor data acquired by an arbitrary number of sensors or sensor devices. For example, instead of only analyzing a single type (e.g., dimension) of the objective data (e.g., sensor data), an additional type of objective data (e.g., blood pressure data) or subjective data (e.g., a desired outcome or evaluation) can be included. In this way, any number or types of data can act as discrete inputs to the compatibility algorithm 130. In implementations where the compatibility algorithm 130 is a neural network or deep belief network, each type of data can be the input to an input node.

To determine an individual's compatibility with a group, or to compare an individual's response to that of a group, the corresponding sensor data of the individuals in the group can be aggregated. For example, if a user's response to a stimulus is different than that of the typical member of a potential group, then the system (in some implementations using a machine-learning algorithm) can predict that the user is not compatible with the group. In this way, by providing a measure of an individual's compatibility with the group, the overall dynamics of the group can be improved by the potential exclusion or discouragement of participation by users that are determined to likely have a lower compatibility with the existing group. By low compatibility, this can describe a user whose compatibility score is below, for example, an average of the group when considering events that the group participates in, or the user having a compatibility score with the group that is below a predetermined threshold.

The aggregated sensor data can be joined in a tabular format or otherwise appended to pre-existing data entries. In other implementations, the aggregated data can be a processed subset of the sensor data. For example, the aggregated data can be an average, sum, difference, or the like of different groups of sensor data. The aggregation can be performed in any order. For example, the raw data can be aggregated and then a single compatibility score 120 generated. In another implementation, the compatibility score 120 between the user and each member of the group can be calculated and then an average compatibility score 120 can be provided based on the individual scores.

Through a user-interface, a user can select a collection of other users as desired groups. Desired groups can include, for example, emotionally desired groups, which the user can select based on attraction, and rationally desired groups, which the user can select based on a rational belief that such members are the correct matches for them. The user can define the measures (or aspects) of the emotionally desired groups based on, for example, physical appearance, hobbies, personality types, etc. Similarly, the user can define the measures of the rationally desired group selection based on such priorities as, for example, security, love, protection, money, morality, religion, politics, etc.

The system can create a matched group for the user based on the user's desired groups and the harmony and correlation analysis of the user's sensor data and sensor data for the desired group.

In some implementations, the system can provide one or more matches between a user and one or more members of a group. A match can include, for example, a best match (e.g., the highest compatibility score 120 with a member of the group) or a group of matches (e.g., the members of the group that have a compatibility score 120 with the user that exceeds a certain threshold). The threshold for matching can be set by the system, entered by the user, or also defined by one or more members of the group. Any combination of metrics for matching or generating a compatibility score 120 can be used. For example, matching or compatibility can be based on raw data, processed sensor data (e.g., including tonic and/or phasic data), aggregated data of any type, or the like. Matching can also be based on a comparison of the compatibility score 120 between a first user and a particular group and the compatibility score 120 between a second user and the particular group. For example, if one user is very compatible with a particular group, but a second user is not compatible with the same group, then the system can avoid matching the first user with the second user. Such cross-user matching can act as a constraint when generating a final compatibility score 120 for a user with the compatibility algorithm 130.

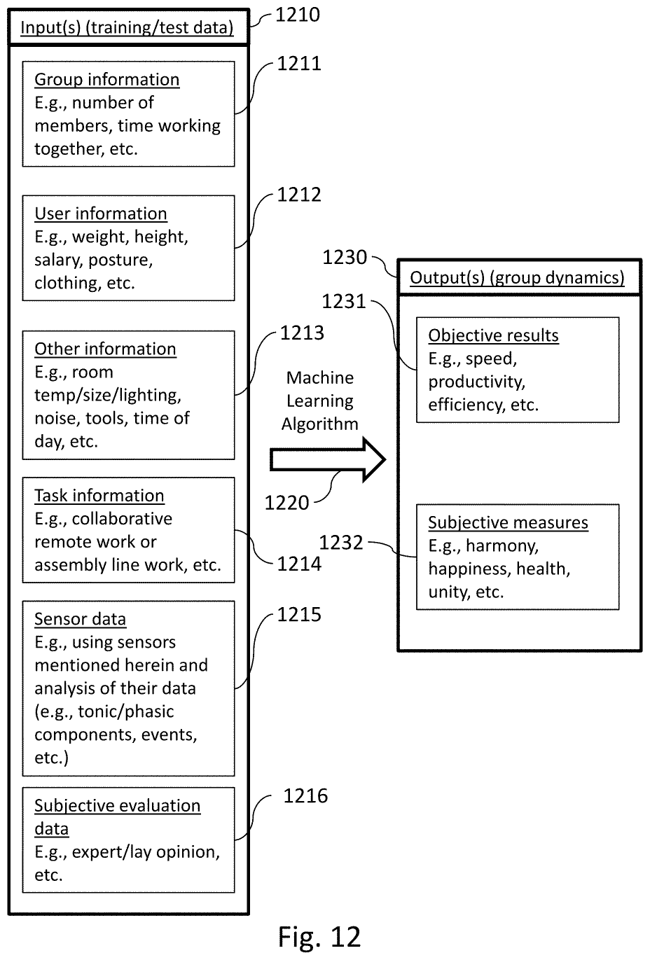

FIG. 12 illustrates a simplified system for generating and presenting a quantified group dynamic determined by a machine learning system in accordance with certain aspects of the present disclosure. Group dynamics can be quantified in a manner similar to compatibility—previously described as a metric indicative of the compatibility or harmony between the two or more individuals. Accordingly, many of the features of the previously disclosed embodiments can be utilized for embodiments that quantify group dynamics. For example, previously disclosed embodiments can be generally characterized as determining compatibility, based on sensor data and optionally subjective data, of a one-to-one group (e.g., a first user and a second user) or compatibility of a one-to-many group (e.g., a first user and a group). Such embodiments can also be utilized for similar evaluations of how group dynamics may change in response to changes in a group. This can be akin to a system analyzing a change in a many-to-many system (e.g., changing one defined group to another, different, defined group). Such changes can include, e.g., adding/removing members, changing attributes of group members, changes in the environment where the group is located, etc.

Some embodiments of the present disclosure can include computer-implemented methods of training a machine learning algorithm to determine a quantification of a group dynamic based on input about the group. The accuracy of the quantified group dynamics can be improved by also training the machine learning algorithm with subjective evaluation data (e.g., from a human expert or layperson observing the group). The trained machine learning algorithm can then generate predictions of group dynamics based on a proposed group or changes to an existing group. As one example, during a training process, a group of three people may have been determined to have high compatibility (i.e., positive group dynamics) by the machine learning algorithm performing an analysis of their sensor data, group attributes, and subjective evaluation data over a wide range of tasks, environments, etc. The trained machine learning algorithm can then predict what the change to the group dynamics would be were the to be group changed (e.g., by increasing the number of members to ten). As a more specific example, the machine learning algorithm can determine that by changing the number of members from 3 to 10, the speed of completion of a particular task increased from two tasks per hour to five tasks per hour. As explained further herein by numerous examples, the present disclosure contemplates that the machine learning algorithm can be utilized to analyze a wide variety of groups, in a wide variety of situations, and provide a broad range of quantified group dynamics. As with other embodiments disclosed herein, the quantified group dynamic can be provided to client devices such as computers, smartphones, etc., for electronic display in various formats (text, graphical, etc.).

As shown in the block diagram of FIG. 12, some embodiments can include computer software that, at 1210, can receive input data related to a particular group. At 1220, the machine learning algorithm can determine a quantified group dynamic for the particular group. As described further herein, the machine learning algorithm can be trained with such exemplary input data including one or more of group information, user information, sensor data, subjective evaluation data, etc. At 1230, a client device can generate an electronic indication of the quantified group dynamic. Additional details of the various elements depicted in FIG. 12 are provided below.

In some embodiments, the evaluation of an expert will be used to guide the training of the machine learning algorithm and thereby serve as the desired output. For example, input data can be given to the machine learning algorithm and the learning iterations and the coefficients in the layers of the machine learning algorithm can be trained so that the machine learning algorithm output approximates (or matches) to the multi-dimensional assessments of the expert evaluator. A certain percentage of the training data (e.g., 20%, 10%, 5%, etc.) can be kept as testing data to avoid overfitting of the machine learning algorithm.

The quantified group dynamic 1230 can include, for example, objective results 1231 including one or more of: task completion speed, group productivity, or group efficiency. Such objective results can be determined based on facts, observations, or other input data (e.g., start/stop times) that do not require subjective analysis. For example, the machine learning algorithm can output (e.g., causing display at a client device) an electronic indication that a group productivity is three tasks per hour. Similarly, by modifying the input to the machine learning algorithm, the outputted quantified group dynamic could change to ten tasks per hour thereby indicating the positive effect of the modified group.

Similarly, in some embodiments, the quantified group dynamic 1230 can include subjective measures 1232 including one or more of group health, group happiness, group unity, or learning. Subjective measures can be those that are not readily quantifiable by measurable group data but can still be predicted by the trained machine learning algorithm. For example, the machine learning algorithm can be trained to calculate the happiness (which may be quantified on a numerical scale). Similar to calculating objective results, a change in the input (e.g., a change to the group) to the machine learning algorithm can then allow it to calculate the happiness of the changed group. The subjective measures can be output in a manner similar to an objective result, e.g., displaying at a client device that the group happiness has (or would) change from 20/100 to 90/100. As another example, the subjective measure of learning can be assessed subjectively by an expert but, in some embodiments, can also be treated as an objective result based on tests taken by the group.

Some embodiments can perform ongoing analysis of a group based on ongoing monitoring of features of the group based on changing input data about the group. For example, this can include receiving updated input data such as adding/removing a group member, a change in an attribute of a group member such as salary, receiving continuing/ongoing sensor data/feedback, etc. The machine learning algorithm can then determine an updated quantified group dynamic. The software can generate, at the client device, an updated electronic indication of the updated quantified group dynamic. In some embodiments, the system can be used to evaluate the evolution of a group over time (where any of the types of data contemplated herein can be provided as updated input data). In this way, the system can be used for maintenance of various aspects of group dynamics. For example, various family members could compare their biofeedback data and learn about the various aspects of their family after they themselves train the system based on their own subjective evaluation of the output.

The present disclosure contemplates that any suitable input data, sensor data, subjective evaluation data, etc., can be utilized by the machine learning algorithm. Some nonlimiting examples of such data are provided below but are not intended to be an exhaustive list. Accordingly, a person of skill would be able to utilize the present disclosure with other types of data (or specific examples of such) that can be provided to the machine learning algorithm.

In some embodiments, the input data can include group information 1211 including, for example, a number of members, a length of time the group has been working together, etc. The input data can also include user information 1212 including height, weight, education level, salary, posture, type of clothing worn (e.g., professional or casual dress), etc. The sensor data 1215 can include, for example, biofeedback data from a sensing device, such as a galvanic skin sensors, photoplethysmography sensors, etc., as described herein.

The input data can further include subjective evaluation data 1216 including an evaluated group dynamic determined based at least on the sensor data. As previously described, subjective evaluation data can include quantification by a human user (expert or layperson) or observer that can interpret observed or quantified group dynamics that may require subjective analysis. For example, the subjective evaluation data can include inputting an expert's subjective evaluation of the happiness of a particular group doing a particular task. Such subjective evaluation data can compliment the analysis of biofeedback data used by the machine learning algorithm to quantify the group dynamics. In another example, the expert may provide his or her subjective evaluation following a review of the sensor data, or review of a decomposition of the biofeedback data into its tonic and/or phasic components and then, based on their expert knowledge, assess the happiness, harmony, etc., of the group. In this way, the quantified group dynamic output by the machine learning algorithm can be synergistically more accurate than it would be were the objective input (e.g. group information, user information, etc.) and subjective input (e.g. the subjective evaluation data) used alone.

In various embodiments, the input data can include other information 1213 such as a room temperature, a room size, a time of day, or a noise level. The input data can also include task information 1214 including, for example, collaborative remote work, assembly line work, etc. Task information can include information such as naming the task, parameters of the task, (e.g., location, duration, complexity, etc.), average duration, etc. Other examples of tasks that can be performed by users as part of training or using the machine learning algorithm (and that have corresponding task information to serve as input data) can include such things as administrative tasks in the same small space (e.g., data entry, programming, providing telephone support, sales calls, providing medical/mental support or advice, accounting, design, classroom activities etc.), factory work such as on an assembly line or in a manufacturing space, learning a subject, workers or students being challenged to solve a problem or take a test, working in a retail context (doing sales or providing support), providing food service, or tasks remotely done by workers collaborating or working on a similar tasks together at the same time. Tasks may also be performed at different times, for example, if a group of people (A) work on a task at time X and pass the work to another set of individuals (B) at time Y. The data gathered at time X and Y data could be analyzed similar to the way data is analyzed concurrently.

In some embodiments, the sensor data can be from a galvanic skin sensor, a nano-sensor inserted in the body (which may also include inserting nano-sensors inserted in the skin, e.g., a between 10 and 2000 microns deep), EKG, EEG, fMRI (or other types of sensors that measure functions of the body and/or brain), or any of the other types of sensors and their associated sensor data disclosed herein (e.g., sweat, blood pressure, heart rate, breath, saliva, urine, etc.). Similarly, various embodiments can include sensing devices that generate the sensor data, such as contained in a watch or wristband, a smart-ring, or any of the other types of sensing devices disclosed herein.

Again, previously described aspects of the present disclosure can be included in embodiments that describe analysis performed by the machine learning algorithm. For example, in some embodiments, software can determine an event window from the sensor data. An attribute can be added to the sensor data in the event window and the attribute can be included with the input data. As one example, the biofeedback data can be analyzed with various peaks or other features of the data identified via software (e.g., such as exceeding a particular threshold). Such identifications (e.g., a greeting, a dispute, etc.) can then be labeled by the software, or by a human user, as a particular event. Such classifications of sensor data can facilitate the accurate determination of a group dynamic by the machine learning algorithm.

A specific example of the use of the trained machine learning algorithm is described here. This example is for quantifying a change in group dynamics where the particular group differs in a group size or a group location from a training group from which the group information, the user information, the sensor data, and the subjective evaluation data were obtained. In this simplified example, the training group(s) can include groups having between 10, 20, 50, and 100 members. The training group(s) can be observed during actual work or other activities (in their normal locations) or located in a variety of locations (e.g., an office setting, a restaurant, a social gathering, etc.). This information about the group can be utilized as group information with particular attributes of members of the group utilized as user information. Some or all of the training group(s) can be outfitted with sensors to obtain sensor data. The group dynamics can be observed by an evaluator to provide subjective evaluation data that can be associated with, or used to classify, characterize, or label, the sensor data. The above data can then be used to train the machine learning algorithm to accurately provide objective results (e.g., the speed at which a task is performed by the various group(s)) and/or subjective measures (e.g., the harmony, happiness, health, etc. of the various group(s)).

With the trained machine learning algorithm, similar input data can be provided but for a different particular group (e.g., a group with 30 members in a restaurant setting). The input data for the 30 member group can be provided similarly to that provided for the training groups (e.g., inputting group information, user information, etc.). The machine learning algorithm can then output quantified group dynamics that would accurately describe group dynamics for the 30 member group. For example, it can predict that such a group would have a particular productivity and a particular happiness level when working together.

In some embodiments, the system can be configured to perform an ongoing analysis of the input data to generate the quantified group dynamic. For example, the sensor data can be obtained as timeseries data as described herein. Similarly, other input data can also be acquired in an ongoing manner such as group/user location, room temperature, user posture, the subjective evaluation (which could change over time), etc. In particular embodiments, the input data can be received simultaneously with the ongoing analysis. For example, in real-time, or nearly so, the instant status of the group can be provided to the machine learning algorithm as input data and received/updated simultaneously with the ongoing analysis that generates the quantified group dynamic. In this way, the client device can display the current (and possibly evolving) quantified group dynamic to show the real-time changes in the group dynamic responsive to changes in the input data about the group.

In some embodiments, the disclosed software can perform additional training of the machine learning algorithm during use of the machine learning algorithm. Periods of use can include a time when receiving the input data or determining the quantified group dynamic but may also include other times when additional training data is provided to the machine learning algorithm such as part of a software update, or user-added training data. For example, the additional training of the machine learning algorithm can be based at least on the input data and subsequent subjective evaluation data obtained during use. The subsequent subjective evaluation data may be a complete evaluation, as described previously—or may be a response by a user indicating whether a previous quantified group dynamic was correct.

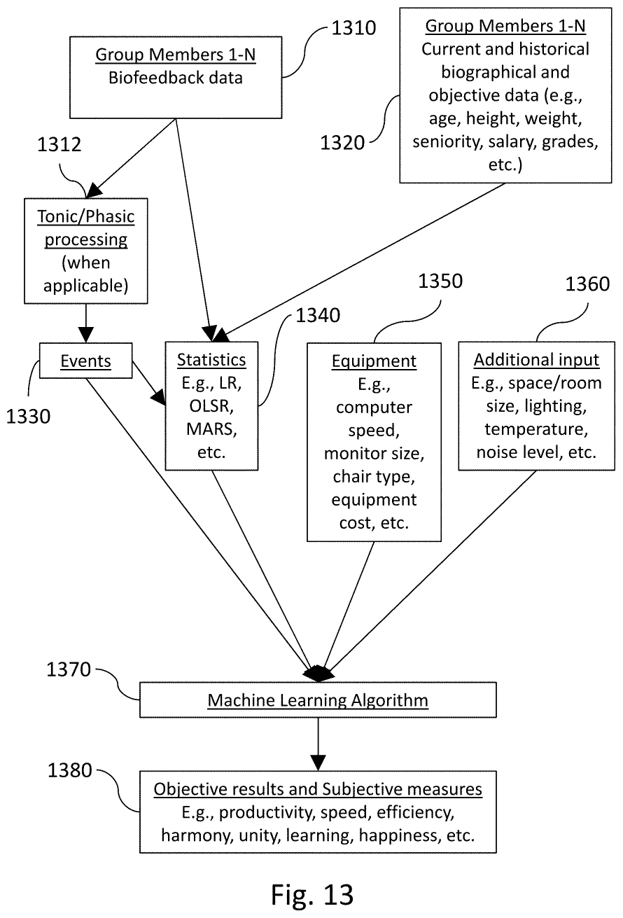

FIG. 13 illustrates an alternative embodiment for generating and presenting subjective measures determined by a machine learning algorithm in accordance with certain aspects of the present disclosure. Biofeedback data for group members (1-N) can be obtained at 1310 (and at 1312, when applicable, processed into tonic/phasic components). Current and/or historical biographical and other objective data (e.g., age, height, weight, seniority, salary, grades, etc.) can be obtained at 1320. At 1330, event detection can be performed on the biofeedback data. At 1340, statistical methods such as LR, OLSR, MARS, etc., can be performed to calculate various statistical characterizations of the data from 1310 and/or 1320. At 1350, information about the equipment used (e.g., computers speed, monitor size, chair type, equipment costs, etc.) can be obtained. At 1360, additional input can include, for example, space/room size, lighting, temperature, noise level, etc., any combination of these inputs (1330, 1340, 1350, 1360) can be input into machine learning algorithm 1370. The machine learning algorithm can then, at 1380, output objective results (e.g., productivity, speed, efficiency, etc.) and subjective measures (e.g., harmony, unity, learning, happiness, etc.) in a manner and format similar to other embodiments disclosed herein.

Two examples of machine learning algorithms that may be implemented in any of the disclosed embodiments can include a neural network or a deep belief algorithm. The systems and methods described herein can include applying one or more machine learning algorithms to, for example, generate a decision, compatibility score 120, or a quantified group dynamic. In some implementations, the machine learning algorithm can be trained with a predefined training data set. In other implementations, the machine learning algorithm can be trained during use by a user providing user input that evaluates the accuracy of the decision or compatibility score 120. Such an approach has been previously discussed with regard to the inputting of user preferences and a user's evaluation of an event.

In some implementations, the machine learning algorithm for processing the input data can be, for example, one or more of: Ordinary Least Squares Regression (OLSR), Linear Regression, Logistic Regression, Stepwise Regression, Multivariate Adaptive Regression Splines (MARS), Locally Estimated Scatterplot Smoothing (LOESS), k-Nearest Neighbor (kNN), Learning Vector Quantization (LVQ), Self-Organizing Map (SOM), Locally Weighted Learning (LWL), Ridge Regression, Least Absolute Shrinkage and Selection Operator (LASSO), Elastic Net, Least-Angle Regression (LARS), Classification and Regression Tree (CART), Iterative Dichotomiser 3 (ID3), C4.5 and C5.0 (different versions of a powerful approach), Chi-squared Automatic Interaction Detection (CHAID), Decision Stump, M5, Conditional Decision Trees, Naive Bayes, Gaussian Naive Bayes, Multinomial Naive Bayes, Averaged One-Dependence Estimators (AODE), Bayesian Belief Network (BBN), Bayesian Network (BN), k-Means, k-Medians, Expectation Maximisation (EM), Hierarchical Clustering, Apriori algorithm, Eclat algorithm, Perceptron, Back-Propagation, Hopfield Network, Radial Basis Function Network (RBFN), Deep Boltzmann Machine (DBM), Deep Belief Networks (DBN), Convolutional Neural Network (CNN), Stacked Auto-Encoders, Boosting, Bootstrapped Aggregation (Bagging), AdaBoost, Stacked Generalization (blending), Gradient Boosting Machines (GBM), Gradient Boosted Regression Trees (GBRT), and Random Forest.

The raw or processed forms of a user's sensor data, or any compatibility scores 120, can be made available to the user by presentation on, for example, a client device 150 (e.g., smartphone, desktop, laptop, or tablet computer), the local device (used to relay the sensor data), the sensor device, or the like. The sensor data can also be presented to other users that have obtained the necessary electronic permissions. Some implementations of the system described herein can include a permission granting system that allows a user to grant and revoke permission for other users to view and or access their raw or processed sensor data. The permission granting system can have multiple levels of access to sensor data or aggregate data of other interested parties.

In some implementations, the system can receive permission settings entered at an input device by the first user. The system can then restrict, based at least on the permission settings, access by the second user to the sensor data or to the analyzed sensor data generated by the compatibility algorithm 130 when generating the compatibility score 120. In other implementations, the permission settings can be timed to expire at a certain date or time, or remain for a specified length of time before reverting back to a prior, or default, setting.

Permission settings can include, for example, restricting the ability of a second user to view, copy, edit, delete, or the like, any or all of the first user's sensor data or compatibility scores or analysis. Conversely, the user can allow progressively increasing levels of access to a second user based on the level of trust or relationship status between the first user and the second user. In some implementations the permission settings can be automatically adjusted based on a current compatibility score 120.

In the following, further features, characteristics, and exemplary technical solutions of the present disclosure will be described in terms of items that may be optionally claimed in any combination:

Item 1: A computer program product comprising a non-transient, machine-readable medium storing instructions which, when executed by at least one programmable processor, cause the at least one programmable processor to perform operations comprising receiving input data related to a particular group and determining, by a machine learning algorithm, a quantified group dynamic for the particular group. The machine learning algorithm can be trained with at least group information, user information, sensor data, and subjective evaluation data. A client device can generate an electronic indication of the quantified group dynamic.

Item 2: The computer program product of any one of the preceding items: where the quantified group dynamic can include objective results including one or more of task completion speed, group productivity, or group efficiency.

Item 3: The computer program product of any one of the preceding items: where the quantified group dynamic comprises subjective measures can include one or more of group health, group happiness, or group unity.

Item 4: The computer program product of any one of the preceding items: the operations including receiving, at the at least one programmable processor, updated input data, determining, by the machine learning algorithm, an updated quantified group dynamic, and generating, at the client device, an updated electronic indication of the updated quantified group dynamic.

Item 5: The computer program product of any one of the preceding items: where the receiving of the updated input data can include receiving ongoing sensor data.

Item 6: The computer program product of any one of the preceding items: where input data can include group information including one or more of a number of members or a length of time the group has been working together.

Item 7: The computer program product of any one of the preceding items: where the input data can include comprises user information including one or more of height, weight, or education level.

Item 8: The computer program product of any one of the preceding items: where the sensor data can include biofeedback data from a sensing device.

Item 9: The computer program product of any one of the preceding items: where the input data can include subjective evaluation data including an evaluated group dynamic determined based at least on the sensor data.

Item 10: The computer program product of any one of the preceding items: where the input data can include one or more of a room temperature, a room size, a time of day, or a noise level.

Item 11: The computer program product of any one of the preceding items: where the input data can also include task information including one or more of collaborative remote work or assembly line work.

Item 12: The computer program product of any one of the preceding items: where the sensor data can be from a galvanic skin sensor a nano-sensor inserted in the skin, EKG sensor, EEG sensor, temperature sensor, or fMRI.

Item 13: The computer program product of any one of the preceding items: where a sensing device that generates the sensor data can be contained in a watch or wristband.

Item 14: The computer program product of any one of the preceding items: the operations including determining an event window from the sensor data and adding an attribute to the sensor data in the event window, where the attribute is included with the input data.

Item 15: The computer program product of any one of the preceding items: where the particular group differs in one or more of a group size or group location from a training group from which the group information, the user information, the sensor data, and the subjective evaluation data was obtained.

Item 16: The computer program product of any one of the preceding items: where the operations can include performing an ongoing analysis of the input data to generate the quantified group dynamic, where the input data is received simultaneously with the ongoing analysis.

Item 17: The computer program product of any one of the preceding items: where additional training of the machine learning algorithm can be performed during use of the machine learning algorithm when receiving the input data or determining the quantified group dynamic, the additional training based at least on the input data and subsequent subjective evaluation data.

Item 18: The computer program product of any one of the preceding items: where the machine learning algorithm can include one or more of a neural network or a deep belief algorithm.

Item 19: A method comprising the operations of any one of the preceding Items.

Item 20: A system comprising: at least one programmable processor; and a non-transitory machine-readable medium storing instructions which, when executed by the at least one programmable processor, cause the at least one programmable processor to perform operations comprising those of any one of items 1-18.

Implementations of the current subject matter can include, but are not limited to, systems and methods consistent with the descriptions provided herein as well as articles that comprise a tangibly embodied machine-readable medium operable to cause one or more machines (e.g., computers, etc.) to result in operations implementing one or more of the described features. Similarly, computer systems are also contemplated that may include one or more processors and one or more memories coupled to the one or more processors. A memory, which can include a computer-readable storage medium, may include, encode, store, or the like, one or more programs that cause one or more processors to perform one or more of the operations described herein. Computer implemented methods consistent with one or more implementations of the current subject matter can be implemented by one or more data processors residing in a single computing system or across multiple computing systems. Such multiple computing systems can be connected and can exchange data and/or commands or other instructions or the like via one or more connections, including but not limited to a connection over a network (e.g., the internet, a wireless wide area network, a local area network, a wide area network, a wired network, or the like), via a direct connection between one or more of the multiple computing systems, etc.

The details of one or more variations of the subject matter described herein are set forth in the accompanying drawings and the description. Other features and advantages of the subject matter described herein will be apparent from the description and drawings. While certain features of the currently disclosed subject matter are described for illustrative purposes in relation to particular implementations, it should be readily understood that such features are not intended to be limiting.

Although a few embodiments have been described in detail above, other modifications are possible. For example, the method steps depicted in FIGS. 3-5 and described herein do not require the particular order shown, or sequential order, to achieve desirable results.

The present disclosure contemplates that the calculations disclosed in the embodiments herein may be performed in a number of ways, applying the same concepts taught herein, and that such calculations are equivalent to the embodiments disclosed.

One or more aspects or features of the subject matter described herein can be realized in digital electronic circuitry, integrated circuitry, specially designed application specific integrated circuits (ASICs), field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to programs, software, software applications, applications, components, or code, include machine instructions for a programmable processor, and can be implemented in a high-level procedural language, an object-oriented programming language, a functional programming language, a logical programming language, and/or in assembly/machine language. As used herein, the term “machine-readable medium” (or “computer readable medium”) refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” (or “computer readable signal”) refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including, but not limited to, acoustic, speech, or tactile input. Other possible input devices include, but are not limited to, touch screens or other touch-sensitive devices such as single or multi-point resistive or capacitive trackpads, voice recognition hardware and software, optical scanners, optical pointers, digital image capture devices and associated interpretation software, and the like.

In the description, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. The implementations described above can be directed to various combinations and subcombinations of the disclosed features and/or combinations and subcombinations of further features noted above. Furthermore, above-described advantages are not intended to limit the application of any issued claims to processes and structures accomplishing any or all of the advantages.

Additionally, section headings shall not limit or characterize the invention(s) set out in any claims that may issue from this disclosure. Specifically, and by way of example, although the headings refer to a “Technical Field,” such claims should not be limited by the language chosen under this heading to describe the so-called technical field. Further, the description of a technology in the “Background” is not to be construed as an admission that technology is prior art to any invention(s) in this disclosure. Multiple inventions may be set forth according to the limitations of the multiple claims issuing from this disclosure, and such claims accordingly define the invention(s), and their equivalents, that are protected thereby.

1. A computer program product comprising a non-transient, machine-readable medium storing instructions which, when executed by at least one programmable processor, cause the at least one programmable processor to perform operations comprising:
receiving, at the at least one programmable processor, input data related to a particular group;
determining, by a machine learning algorithm, a quantified group dynamic for the particular group, the machine learning algorithm trained with at least group information, user information, sensor data, and subjective evaluation data; and
generating, at a client device, an electronic indication of the quantified group dynamic.

2. The computer program product of claim 1, wherein the quantified group dynamic comprises objective results including one or more of task completion speed, group productivity, or group efficiency.

3. The computer program product of claim 1, wherein the quantified group dynamic comprises subjective measures including one or more of group health, group happiness, or group unity.

4. The computer program product of claim 1, the operations further comprising:
receiving, at the at least one programmable processor, updated input data;
determining, by the machine learning algorithm, an updated quantified group dynamic; and
generating, at the client device, an updated electronic indication of the updated quantified group dynamic.

5. The computer program product of claim 4, wherein the receiving of the updated input data comprises receiving ongoing sensor data.

6. The computer program product of claim 1, wherein the input data further comprises group information including one or more of a number of members or a length of time the group has been working together.

7. The computer program product of claim 1, wherein the input data further comprises user information including one or more of height, weight, or education level.

8. The computer program product of claim 1, wherein the sensor data comprises biofeedback data from a sensing device.

9. The computer program product of claim 1, wherein the input data further comprises subjective evaluation data including an evaluated group dynamic determined based at least on the sensor data.

10. The computer program product of claim 1, wherein the input data further comprises one or more of a room temperature, a room size, a time of day, or a noise level.

11. The computer program product of claim 1, wherein the input data further comprises task information including one or more of collaborative remote work or assembly line work.

12. The computer program product of claim 1, wherein the sensor data is from a galvanic skin sensor a nano-sensor inserted in the skin, EKG sensor, EEG sensor, temperature sensor, or fMRI.

13. The computer program product of claim 1, wherein a sensing device that generates the sensor data is contained in a watch or wristband.

14. The computer program product of claim 1, the operations further comprising:
determining an event window from the sensor data; and
adding an attribute to the sensor data in the event window, wherein the attribute is included with the input data.

15. The computer program product of claim 1, wherein the particular group differs in one or more of a group size or group location from a training group from which the group information, the user information, the sensor data, and the subjective evaluation data was obtained.

16. The computer program product of claim 1, the operations further comprising performing an ongoing analysis of the input data to generate the quantified group dynamic, wherein the input data is received simultaneously with the ongoing analysis.

17. The computer program product of claim 1, the operations further comprising performing additional training of the machine learning algorithm during use of the machine learning algorithm when receiving the input data or determining the quantified group dynamic, the additional training based at least on the input data and subsequent subjective evaluation data.

18. The computer program product of claim 1, wherein the machine learning algorithm comprises one or more of a neural network or a deep belief algorithm.

19. A system comprising:
at least one programmable processor; and
a non-transient, machine-readable medium storing instructions which, when executed by at least one programmable processor, cause the at least one programmable processor to perform operations comprising:
receiving, at the at least one programmable processor, input data related to a particular group;
determining, by a machine learning algorithm, a quantified group dynamic for the particular group, the machine learning algorithm trained with at least group information, user information, sensor data, and subjective evaluation data; and
generating, at a client device, an electronic indication of the quantified group dynamic.
